(19) United States
(12) Patent Application Publication
(10) Pub. No.: US 2014/0036636 A1
(43) Pub. Date: Feb. 6, 2014

(54) OBJECT INFORMATION ACQUIRING APPARATUS AND OBJECT INFORMATION ACQUIRING METHOD

(71) Applicant: CANON KABUSHIKI KAISHA, Tokyo (JP)

(72) Inventor: Takuro Miyasato, Kyoto-shi (JP)

(73) Assignee: CANON KABUSHIKI KAISHA, Tokyo (JP)

(21) Appl. No.: 13/950,506

(22) Filed: Jul. 25, 2013

(30) Foreign Application Priority Data
Aug. 1, 2012 (JP) 2012-171050

(51) Int. Cl. A61B 5/00

(52) U.S. Cl. CPC A61B 5/0095
USPC 367/178

(57) ABSTRACT

Employed is an object information acquiring apparatus including an irradiation unit irradiating an object with light; a detection unit including a plurality of elements each obtaining an acoustic wave generated from the object and converting the acoustic wave into a detection signal; a detection unit moving mechanism moving the detection unit to the object relatively; an adding unit selecting, from detection signals converted from acoustic waves obtained by the plurality of elements at respective positions as the detection unit is moved, detection signals converted from acoustic waves obtained from an identical position on the object, and adding together these detection signals to output a resultant summed signal; an initial sound pressure computing unit computing an initial sound pressure of a region of interest from the summed signal; a light quantity computing unit computing a quantity of light of the region of interest.

### BACKGROUND OF THE INVENTION

1. Field of the Invention

The present invention relates to an object information acquiring apparatus and an object information acquiring method.

2. Description of the Related Art

Optical imaging apparatuses for acquiring information of the inside of an object by irradiating the object with light from a light source such as laser and propagating the light through the object are being actively researched mainly in medical fields. Photoacoustic imaging (PAI) is one of such optical imaging techniques. PAI will be explained below.

PAI is a technique of visualizing information related to internal optical properties of a living body (object), wherein the living body (object) is irradiated with pulsed light from a light source, and a photoacoustic wave that is generated when the light is absorbed by living tissues as it propagates and diffuses inside the living body is detected, processed, and analyzed. Through such analysis, distributions of optical properties inside the living body, in particular, initial sound pressure distributions, light energy absorption densities, absorption coefficient distributions, and oxygen saturation distributions, can be obtained.

Initial sound pressure P₀ of a photoacoustic wave generated from a light absorber inside the object can be expressed by the following equation (1):

P₀=Γ·μ_{a·Φ} (1),

where F is a Grueneisen parameter, which is obtained by dividing the product of a volume expansion coefficient β and a squared sound velocity ratio c by a specific heat capacity at constant pressure C_{P}, and known to be substantially constant when the object is specified, μₐ is an absorption coefficient of the light absorber, and Φ is a local quantity of light (quantity of light absorbed by the light absorber, also called “light fluence”).

Japanese Patent Application Laid-open No. 2010-88627 (Patent Literature 1, or PTL 1) discloses a technique of calculating an initial sound pressure distribution from changes with time in the sound pressure P of a photoacoustic wave propagated through an object, which are detected by an acoustic wave detector. According to PTL1, an optical energy absorption density, which is the product of μₐ and Φ, can be obtained by dividing the calculated initial sound pressure distribution by the Grueneisen parameter Γ. To obtain the absorption coefficient μₐ, from the distribution of the initial sound pressure P₀, as can be seen from Equation (1), the quantity of light Φ inside the object needs to be determined. That is, the absorption coefficient can be obtained by dividing the initial sound pressure by the quantity of light.

Japanese Patent Application Laid-open No. 2010-104816 (Patent Literature 2, or PTL2) describes a photoacoustic imaging apparatus with an acoustic wave detector comprised of a plurality of elements, wherein the acoustic wave detector is moved in a direction of arrangement of the elements to detect a photoacoustic wave, and detection signals obtained at the same position relative to the object are added together. According to PTL2, a wider area can be measured without making the receiving system for processing signals received from the acoustic wave detector large and complex and without causing a cost increase, and also the S/N ratio of signals can be improved.
Patent Literature 1: Japanese Patent Application Laid-open No. 2010-88627
Patent Literature 2: Japanese Patent Application Laid-open No. 2010-104816

### SUMMARY OF THE INVENTION

In photoacoustic imaging, however, one problem was that, an absorption coefficient distribution calculated by the method shown in PTL1, with the use of an initial sound pressure distribution based on a signal obtained by adding together the signals detected at the same position relative to the object as shown in PTL2, was not accurate. As a consequence, an accurate oxygen saturation distribution of the inside of an object could not be obtained.

The present invention was made in view of the problem described above, its object being to improve the calculation accuracy of absorption coefficients in photoacoustic imaging.

The present invention provides an object information acquiring apparatus, comprising:

an irradiation unit irradiating an object with light;

a detection unit including a plurality of elements each obtaining an acoustic wave generated from the object irradiated with light and converting the acoustic wave into a detection signal;

a detection unit moving mechanism moving the detection unit to the object relatively;

an adding unit selecting, from detection signals converted from acoustic waves obtained by the plurality of elements at respective positions as the detection unit is moved, detection signals converted from acoustic waves obtained from an identical position on the object, and adding together these detection signals to output a resultant summed signal;

an initial sound pressure computing unit computing an initial sound pressure of a region of interest inside the object from the summed signal;

a light quantity computing unit computing a quantity of light of the region of interest; and

an object information computing unit computing object information using the initial sound pressure and the light quantity, wherein

the light quantity computing unit gives weight to a light quantity in accordance with processing in the adding unit and calculation processing in the initial sound pressure computing unit, to compute a weighted quantity of light.

The present invention also provides an object information acquiring method, comprising the steps of:

irradiating an object with light, by means of an irradiation unit;

obtaining an acoustic wave generated from the object irradiated with light and converting the acoustic wave into a detection signal, by means of a plurality of elements of a detection unit;

moving the detection unit on the object, by means of a detection unit moving mechanism;

selecting, from detection signals converted from acoustic waves obtained by the plurality of elements at respective positions as the detection unit is moved, detection signals converted from acoustic waves obtained from a relatively identical position to the object, and adding together these detection signals to output a resultant summed signal, by means of an adding unit;

computing an initial sound pressure of a region of interest inside the object from the summed signal, by means of an initial sound pressure computing unit;

computing a quantity of light of the region of interest, by means of a light quantity computing unit; and

computing object information using the initial sound pressure and the light quantity, by means of an object information computing unit, wherein

the light quantity computing unit gives weight to a light quantity in accordance with processing in the adding unit and calculation processing in the initial sound pressure computing unit, to compute the quantity of light.

According to the present invention, absorption coefficients can be calculated with better accuracy in photoacoustic imaging.

Further features of the present invention will become apparent from the following description of exemplary embodiments with reference to the attached drawings.

### BRIEF DESCRIPTION OF THE DRAWINGS

FIG. 1 is a schematic view of an object information acquiring apparatus according to one embodiment;

FIG. 2A to FIG. 2E are schematic views for explaining photoacoustic imaging according to the embodiment; and

FIG. 3 is a flowchart of an object information acquiring method.

### DESCRIPTION OF THE EMBODIMENTS

Preferred embodiments of the present invention will be hereinafter described with reference to the drawings. Note, it is not intended to limit the scope of this invention to the specifics given below, and the sizes, materials, shapes, and relative arrangements or the like of constituent components described below should be changed as required in accordance with the configurations and various conditions of the apparatus to which the invention is applied.

The object information acquiring apparatus of the present invention includes an apparatus for obtaining information of an object as image data by utilizing the photoacoustic effect, wherein the object is irradiated with light (electromagnetic wave) and an acoustic wave thereby generated inside the object is received. The object information thus acquired includes source distributions of acoustic waves generated by irradiation, initial sound pressure distributions inside the object, or, light energy absorption density distributions, absorption coefficient distributions, or density distributions of tissue-forming substances, which are deduced from the initial sound pressure distributions. The density distribution of substance may be, for example, an oxygen saturation distribution, or a density distribution of oxyhemoglobin or reductive hemoglobin.

The acoustic waves referred to in the present invention are typically ultrasound waves, including elastic waves that are called sound waves, ultrasound waves, or acoustic waves. An acoustic wave generated by the photoacoustic effect is referred to as a “photoacoustic wave”, or a “light-induced ultrasound wave”. An acoustic wave detector receives an acoustic wave generated inside an object.

<General Structure of Apparatus>

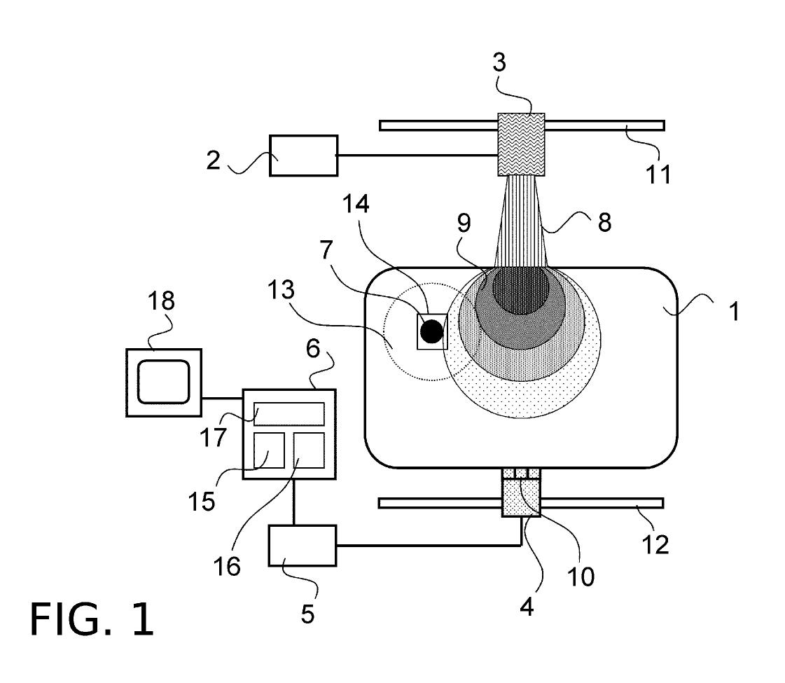

FIG. 1 is a schematic view of an object information acquiring apparatus according to one embodiment. The object information acquiring apparatus in this embodiment includes a light source 2, a light irradiation unit 3, an acoustic wave detector 4, a signal adding and processing unit 5, a computing unit 6, a light irradiation unit moving mechanism 11, an acoustic wave detector moving mechanism 12, and a display device 18. The acoustic wave detector 4 includes acoustic wave detecting elements 10. The computing unit 6 includes an initial sound pressure acquiring module 15, a light quantity acquiring module 16, and an object information acquiring module 17. The measurement target is an object 1, which contains a light absorber 7 inside that has a higher absorption coefficient than its surroundings.

The light source 2 emits pulsed light. The emitted pulsed lights are guided into the light irradiation unit 3, and irradiated onto the object 1 as light beams 8. The light 8 that has entered the object attenuates by diffusion and absorption inside the object (or living tissues if the object is a living body), and thus there is a distribution of light quantity 9 in accordance with the distance from the irradiation position and other factors. When the irradiation light 8 reaches the light absorber 7, a photoacoustic wave 13 is generated by the photoacoustic effect. The light irradiation unit corresponds to the irradiation unit of the present invention.

The generated photoacoustic wave 13 propagates through the object and is detected by the acoustic wave detector 4. More specifically, the photoacoustic wave is received by acoustic wave detecting elements 10a, 10b, and 10c in the acoustic wave detector 4, converted into an analog electrical signal, and output. As the acoustic wave detector 4 of this embodiment includes a plurality of acoustic wave detecting elements, it can detect the photoacoustic wave at a plurality of positions. The signal adding and processing unit 5 amplifies the electrical signal and converts it into a digital signal. The signal adding and processing unit 5 performs averaging on several signals detected at the same position relative to the object 1 and stores the result as a detection signal in a memory (not shown) of the computing unit 6. The acoustic wave detector corresponds to the detector of the present invention.

The initial sound pressure acquiring module 15 as means of acquiring an initial sound pressure in the computing unit 6 acquires an initial sound pressure of a region of interest 14 inside the object 1. The light quantity acquiring module 16 as means of acquiring a quantity of light in the computing unit 6 acquires a quantity of light in the region of interest 14 based on the profile of the irradiation light 8 and an average optical coefficient of the object 1. The object information acquiring module 17 as means of acquiring object information in the computing unit 6 acquires an optical property value of the region of interest 14 based on the initial sound pressure and the light quantity of the region of interest 14. The module 17 then converts the acquired optical property value into image data that can be displayed on the display device 18 provided as display means.

An initial sound pressure distribution inside the object can be obtained by setting regions of interest over the entire area of the object 1 by means of the computing unit 6, and by acquiring the initial sound pressures of all the regions of interest of the whole object by means of the initial sound pressure acquiring module 15. Similarly, a light quantity distribution and an object information distribution may be obtained by acquiring the quantities of light and absorption coefficients of all the regions of interest of the whole object by means of the light quantity acquiring module 16 and the object information acquiring module 17. The region of interest may be referred to as a pixel in two-dimensional image reconstruction, or a voxel in three-dimensional image reconstruction. The initial sound pressure acquiring module, the light quantity acquiring module, and the object information acquiring module correspond to the initial sound pressure computing unit, the light quantity computing unit, and the object information computing unit of the present invention, respectively.

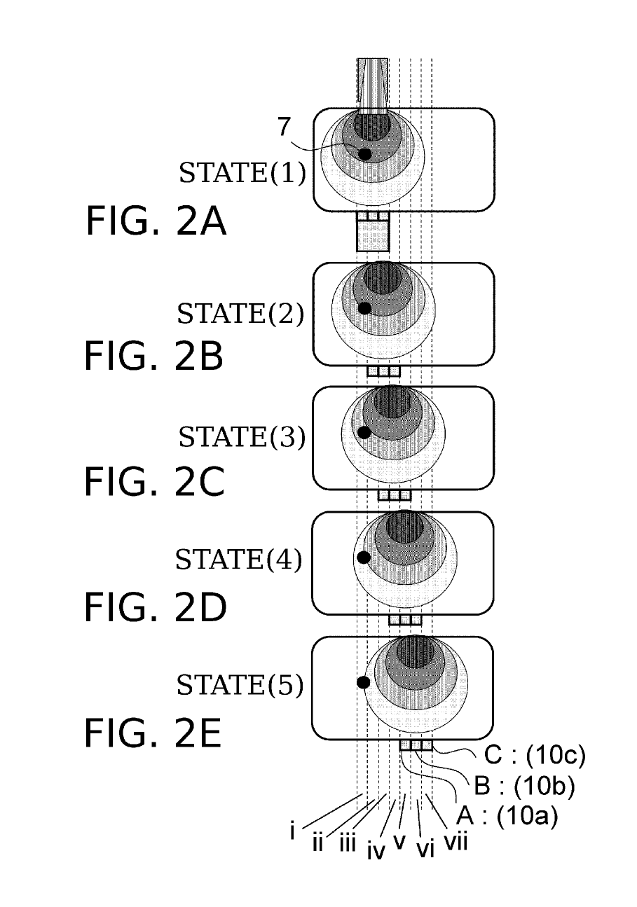

The acoustic wave detector moving mechanism 12 moves the object 1 and the acoustic wave detector 4 relative to each other. As the acoustic wave detector moving mechanism 12 moves the acoustic wave detector 4 including the three acoustic wave detecting elements 10a, 10b, and 10c along the object to scan the object, a photoacoustic wave is detected at several positions, or several times at respective positions, as shown in FIG. 2A to FIG. 2E. The acoustic wave detector moving mechanism corresponds to the detector moving mechanism of the present invention.

The light irradiation unit moving mechanism 11 moves the light irradiation unit 3 to change the position of the irradiation light 8 relative to the object. The acoustic wave detector 4 and the light 8 may be moved in synchronism so that the acoustic wave detector 4 can always acquire signals from a region with a high quantity of light, whereby a photoacoustic wave signal with a high S/N ratio can be obtained. The light irradiation unit moving mechanism corresponds to the irradiation unit moving mechanism of the present invention.

<Details of Constituent Parts>

Next, the respective constituent parts of the object information acquiring apparatus mentioned above will be described in more detail with respect to materials, structures, and functions thereof.

(Light Source)

The light source 2 can emit pulsed light of 5 ns to 50 ns. While high power laser is preferable as the light source, light emitting diodes or the like may also be used instead of laser. Various lasers may be used, such as a solid laser, gas laser, dye laser, and semiconductor laser. Ti:sapphire laser with Nd:YAG as a medium, or Alexandrite Laser, which has high power and allows continuous change of wavelengths, is most preferable. Alternatively, a plurality of single wavelength lasers having different wavelengths may be used.

### (Light Irradiation Unit)

Pulsed lights emitted from the light source 2 are processed to have a desired light distribution profile and guided to the object typically by means of optical components such as lenses and mirrors, but can also be transmitted with the use of light waveguide paths such as optical fibers. The light irradiation unit 3 may be, for example, a mirror that reflects light, a lens that focuses, spreads, or changes the form of light, or a diffusion plate that diffuses light. Any of such optical components may be used as long as the pulsed lights emitted from the light source are irradiated onto the object 1 in a desired shape. It is preferable to spread, rather than focus, the light with a lens to increase the irradiated area to a certain extent, in a sense that it is safer for the object and that the region for diagnosis can be enlarged. To move the light 8, as shown in FIG. 1, a light irradiation unit moving mechanism 11 may be provided to the light irradiation unit.

(Acoustic Wave Detector)

The acoustic wave detector 4 (also called a probe or transducer) detects a photoacoustic wave generated on the surface of and inside the object. It converts the detected acoustic wave into an analog electrical signal. Any devices that detect an acoustic wave may be used, such as a transducer that uses the piezoelectric phenomenon, a transducer that uses light resonance, or a transducer that uses capacitance change. To detect the acoustic wave at a plurality of positions, there should preferably be a plurality of acoustic wave detecting elements 10 arrayed in a line (one dimensional) or on a plane (two dimensional) as shown in FIG. 1 and configured to be mechanically movable by means of the acoustic wave detector moving mechanism 12.

(Signal Adding and Processing Unit)

The signal adding and processing unit 5 amplifies the electrical signal received from the acoustic wave detector 4 and converts the analog signal into a digital signal. The signal adding and processing unit may typically be configured with an amplifier, an A/D converter, and a field programmable gate array (FPGA) chip. If a plurality of detection signals are received from the acoustic wave detector, these signals should preferably be processed at the same time. This will shorten the time before an image is formed. In the present invention, furthermore, the acoustic wave signals detected at the same position relative to the object are added together into one signal. The adding process may be simply adding together the signals, or adding together the signals and dividing the sum by the number of signals, or weighting each signal and adding them together. The term “detection signal” is used herein to refer to a concept including both the analog signal output from the acoustic wave detector and the digital signal converted from this analog signal. The signal adding and processing unit corresponds to the adding unit of the present invention.

(Computing Unit)

The computing unit 6 acquires optical property values of the inside of the object through image reconstruction and the like. Typically, a work station or the like may be used as the computing unit 6, with pre-programmed software to perform the image reconstruction processing. The computing unit can execute various calculation processes. For example, the software used in the work station may include a setting module, the initial sound pressure acquiring module 15, light quantity acquiring module 16, and object information acquiring module 17. The respective modules may be provided as separate hardware. In this case, all the hardware may be referred to collectively as the computing unit 6. In some cases the signal adding and processing unit 5 and the computing unit 6 may be integrated. In this case, the optical property values of the object may be generated through hardware processing instead of the software processing performed in the work station.

<Signal Detection and Adding Process>

Next, a method of acquiring object information according to this embodiment, wherein signals are detected based on light irradiation and the detected signals are weighted with respect to light quantity and added together, will be described, in comparison to a case where the present invention is not applied.

Hereinafter, the relative positional relationship between the object 1 and light absorber 7, and the acoustic wave detector 4 and acoustic wave detecting elements 10a to 10c, when the object is scanned as shown in FIG. 2A to FIG. 2E will be referred to as “state”. FIG. 2A to FIG. 2E correspond to State (1) to State (5), respectively. To distinguish the respective states, superscripts of the state numbers 1 to 5 will be used in the following description. To distinguish the respective acoustic wave detecting elements, subscripts of A to C will be used for the acoustic wave detecting elements 10a to 10c, respectively.

In the following description, the region of interest 14 is set at position r_{T} of the light absorber 7. The light absorber 7 has a higher absorption coefficient for the wavelength of the irradiation light than that of the surrounding tissues so that it generates a photoacoustic wave when irradiated with the light.

State (1) to State (5) respectively have light quantity distributions Φ¹(r), Φ²(r), Φ³(r), Φ⁴(r), Φ⁵(r). The light quantity distribution changes as the light irradiation unit moves in synchronism with the acoustic wave detector. As shown in FIG. 2A to FIG. 2E, the portion with a larger quantity of light moves from left to right on the paper plane.

Detection signals of the region of interest 14 obtained by the acoustic wave detecting elements 10a to 10c in State (1) will be represented by S_{A}¹(r_{T}), S_{B}¹(r_{T}), and S_{C}¹(r_{T}), respectively. Namely, detection signal S_{A}¹(r_{T}) is a signal obtained by the acoustic wave detecting element 10a when the light absorber (region of interest) is located at r_{T}, and the acoustic wave detector is located as it is in State (1). The acoustic wave detecting element 10a is detecting acoustic waves from various directions within the range of its directionality. Thus, based on the distance from this element to the region of interest, and the sound velocity inside the object (living body), the time when an acoustic wave from the region of interest irradiated with light is assumed to have reached the element is calculated, and the signal detected at this time is regarded as the detection signal of the region of interest.

Similarly, the detection signals (electrical signals converted from photoacoustic waves) obtained by the acoustic wave detecting elements 10a to 10c at position r_{T} of the region of interest 14 (light absorber 7) in State (2) are represented by S_{A}²(r_{T}), S_{B}²(r_{T}), and S_{C}²(r_{T}). These signals in State (3) are represented by S_{A}³(r_{T}), S_{B}³(r_{T}), and S_{C}³(r_{T}), in State (4), S_{A}⁴(r_{T}), S_{B}⁴(r_{T}), and S_{C}⁴(r_{T}), and in State (5), S_{A}⁵(r_{T}), S_{B}⁵(r_{T}), and S_{C}⁵(r_{T}).

The signal adding and processing unit 5 adds together sound pressures obtained at the same position on the object to produce a summed signal. The plurality of positions where an acoustic wave may be detected from the object by the acoustic wave detecting elements are referred to as “Position r₍ᵢ₎”, “Position r₍ᵢᵢ₎”, “Position r₍ᵢᵢᵢ₎”, “Position r₍ᵢᵥ₎”, “Position r₍ᵥ₎”, “Position r₍ᵥᵢ₎” and “Position r₍ᵥᵢᵢ₎”, as denoted in FIG. 2A to FIG. 2E. At Position r₍ᵢ₎, only Signal S_{A}¹(r_{T}) is obtained by the acoustic wave detecting element 10a in State (1), so that this signal itself will be the summed signal. At Position r₍ᵢᵢ₎, Signal S_{B}²(r_{T}) is obtained by the acoustic wave detecting element 10b in State (1), and Signal S_{A}²(r_{T}) is obtained by the acoustic wave detecting element 10a in State (2), so that these two signals are added together.

The signal adding and processing unit 5 then computes an average signal. Namely, the unit divides the summed signal obtained as described above by the number of summed signals to determine an average signal. The average signals for respective positions on the object will be denoted as S₍ᵢ₎, S₍ᵢᵢ₎, S₍ᵢᵢᵢ₎, S₍ᵢᵥ₎, S₍ᵥ₎, S₍ᵥᵢ₎, and S₍ᵥᵢᵢ₎. The respective average signals, or the signals averaged for respective positions, of the region of interest 14, are obtained by the following Equations (2) to (8).

S₍ᵢ₎(r_{T})=S_{A}¹(r_{T}) (2)

S₍ᵢᵢ₎(r_{T})=(S_{B}¹(r_{T})+S_{A}²(r_{T}))/2 (3)

S₍ᵢᵢᵢ₎(r_{T})=(S_{C}¹(r_{T})+S_{B}²(r_{T})+S_{A}³(r_{T}))/3 (4)

S₍ᵢᵥ₎(r_{T})=(S_{C}²(r_{T})+S_{B}³(r_{T})+S_{A}⁴(r_{T}))/3 (5)

S₍ᵥ₎(r_{T})=(S_{C}³(r_{T})+S_{B}⁴(r_{T})+S_{A}⁵(r_{T}))/3 (6)

S₍ᵥᵢ₎(r_{T})=(S_{C}⁴(r_{T})+S_{B}⁵(r_{T}))/2 (7)

S₍ᵥᵢᵢ₎(r_{T})=S_{C}⁵(r_{T}) (8)

In the equations, r_{T} represents the position of the light absorber 7.

The positions of the acoustic wave detecting elements are represented by r₍ᵢ₎, r₍ᵢᵢ₎, r₍ᵢᵢᵢ₎, r₍ᵢᵥ₎, r^{(v)}, r₍ᵥᵢ₎, and r₍ᵥᵢᵢ₎.

Correction factors for correcting the respective signals with respect to the positional relationship with the acoustic wave detecting elements are represented by α(r_{T}, r₍ᵢ₎), α(r_{T}, r₍ᵢᵢ₎), α(r_{T}, r₍ᵢᵢᵢ₎), α(r_{T}, r₍ᵢᵥ₎), α(r_{T}, r₍ᵥ₎), α(r_{T}, r₍ᵥᵢ₎), and α(r_{T}, r₍ᵥᵢᵢ₎).

### When the Method of the Present Embodiment is not Applied

The initial sound pressure P₀(r_{T}) at the light absorber 7, if calculated without taking into consideration the processing (such as how many signals were added together) in the signal adding and processing unit 5, will be determined by the following Equation (9).

$${P}_{0}^{n} ( {r}_{T} ) = \{ α ( {r}_{T} , {r}_{( i )} ) {S}_{( i )} ( {r}_{T} ) + α ( {r}_{T} , {r}_{( ii )} ) {S}_{( ii )} ( {r}_{T} ) + α ( {r}_{T} , {r}_{( iii )} ) {S}_{( iii )} ( {r}_{T} ) + α ( {r}_{T} , {r}_{( iv )} ) {S}_{( iv )} ( {r}_{T} ) + α ( {r}_{T} , {r}_{( v )} ) {S}_{( v )} ( {r}_{T} ) + α ( {r}_{T} , {r}_{( vi )} ) {S}_{( vi )} ( {r}_{T} ) + α ( {r}_{T} , {r}_{( vii )} ) {S}_{( vii )} ( {r}_{T} ) \} / 7 \qquad ( 9 )$$

Taking into account that α(r_{T}, r₍ᵢ₎) S_{A}¹(r_{T})=μa (r_{T})·Φ(r_{T}), substituting Equations (2) to (8) into this Equation (9) produces the following Equation (10).

$$\begin{matrix} [ Math . 1 ] & \\ {P}_{0}^{n} ( {r}_{T} ) = {μ}_{a} ( {r}_{T} ) \frac{[ \frac{11}{6} {Φ}^{1} ( {r}_{T} ) + \frac{7}{6} {Φ}^{2} ( {r}_{T} ) + {Φ}^{3} ( {r}_{T} ) + \frac{7}{6} {Φ}^{4} ( {r}_{T} ) + \frac{11}{6} {Φ}^{5} ( {r}_{T} ) ]}{7} & ( 10 )\end{matrix}$$

The light quantity Φ(r_{T}) at the light absorber 7, if calculated without taking into consideration the processing in the signal adding and processing unit 5, will be determined by the following Equation (11).

$$\begin{matrix}[ Math . 2 ] & \\ {Φ}^{n} ( {r}_{T} ) = \frac{{Φ}^{1} ( {r}_{T} ) + {Φ}^{2} ( {r}_{T} ) + {Φ}^{3} ( {r}_{T} ) + {Φ}^{4} ( {r}_{T} ) + {Φ}^{5} ( {r}_{T} )}{5} & ( 11 )\end{matrix}$$

As can be seen, dividing Equation (10) by Equation (11) will not produce an accurate absorption coefficient.

Let us assume now that the absorption coefficient μₐ(r_{T}) of the light absorber 7 is 0.02/mm, and that the light quantities at the light absorber 7 in State (1) to State (5) are: Φ¹(r_{T})=10(J/m²), Φ²(r_{T})=5(J/m²), Φ³(r_{T})=1 (J/m²), Φ⁴(r_{T})=0.3 (J/m²), and Φ⁵(r_{T})=0.05 (J/m²) respectively.

The initial sound pressure P₀ⁿ(r^{T}) of the light absorber 7 at position r₁ is determined as 0.0732 Pa, from Equation (10). However, if the absorption coefficient is calculated based on this initial sound pressure P₀ⁿ(r_{T})=0.0732 Pa and the light quantity Φd (r_{T}) in Equation (11)=3.27 (J/m²), the result becomes 0.0224/mm, which is not accurate.

### When the Method of the Present Embodiment is Applied

In this embodiment, therefore, the average signals for respective positions S₍ᵢ₎, S₍ᵢᵢ₎, S₍ᵢᵢᵢ₎, S₍ᵢᵥ₎, S₍ᵥ₎, S₍ᵥᵢ₎, and S₍ᵥᵢᵢ₎, are weighted before computing an initial sound pressure, based on which a light quantity is calculated, and an absorption coefficient is calculated based on these.

The initial sound pressure of the light absorber 7 at position r_{T} is obtained by Equation (12). The weight is determined in accordance with the number of signals added together in the signal adding and processing unit 5.

$${P}_{0}^{w} ( {r}_{T} ) = \{ α ( {r}_{T} , {r}_{( i )} ) {S}_{( i )} ( {r}_{T} ) + 2 \times α ( {r}_{T} , {r}_{( ii )} ) {S}_{( ii )} ( {r}_{T} ) + 3 \times α ( {r}_{T} , {r}_{( iii )} ) {S}_{( iii )} ( {r}_{T} ) + 3 \times α ( {r}_{T} , {r}_{( iv )} ) {S}_{( iv )} ( {r}_{T} ) + 3 \times α ( {r}_{T} , {r}_{( v )} ) {S}_{( v )} ( {r}_{T} ) + 2 \times α ( {r}_{T} , {r}_{( vi )} ) {S}_{( vi )} ( {r}_{T} ) + α ( {r}_{T} , {r}_{( vii )} ) {S}_{( vii )} ( {r}_{T} ) \} / 7 \qquad ( 12 )$$

Expanding this equation, we find the following Equation (13).

$$\begin{matrix}[ Math . 3 ] & \\ {P}_{0}^{w} ( {r}_{T} ) = {μ}_{a} ( {r}_{T} ) \frac{[ \begin{matrix}3 \times {Φ}^{1} ( {r}_{T} ) + 3 \times {Φ}^{2} ( {r}_{T} ) + 3 \times {Φ}^{3} ( {r}_{T} ) + \\ 3 \times {Φ}^{4} ( {r}_{T} ) + 3 \times {Φ}^{5} ( {r}_{T} )\end{matrix} ]}{7} & ( 13 )\end{matrix}$$

The equation for obtaining the light quantity for computing an accurate absorption coefficient will be as follows (Equation (14)).

$$\begin{matrix}[ Math . 4 ] & \\ {Φ}^{w} ( {r}_{T} ) = \frac{\begin{matrix}3 \times {Φ}^{1} ( {r}_{T} ) + 3 \times {Φ}^{2} ( {r}_{T} ) + 3 \times {Φ}^{3} ( {r}_{T} ) + \\ 3 \times {Φ}^{4} ( {r}_{T} ) + 3 \times {Φ}^{5} ( {r}_{T} )\end{matrix}}{7} & ( 14 )\end{matrix}$$

The initial sound pressure and the light quantity calculated with such weighting are P₀^{w}(r_{T})=0.1401 Pa and Φ^{w}(r_{T})=7.007 (J/m²), respectively. The absorption coefficient is determined as μₐ(r_{T})=0.0200/mm, based on these. Thus the calculation accuracy of the absorption coefficient is improved.

<Processing Procedure>

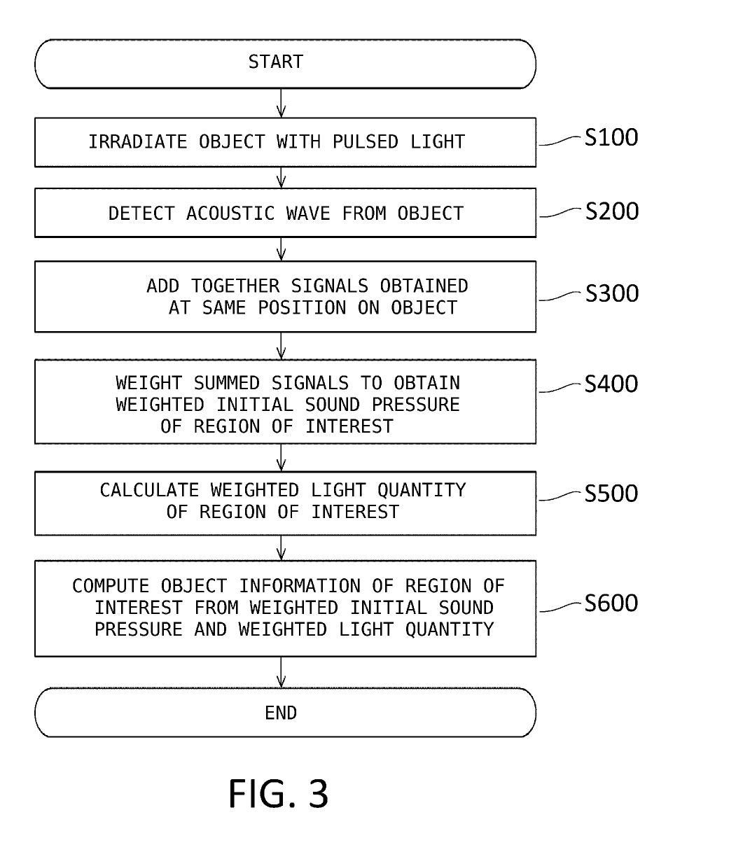

The object information acquiring method according to this embodiment will be described below with reference to the flowchart of FIG. 3. The following reference numerals each correspond to those given to the respective process steps shown in FIG. 3.

(S100: Light irradiation step of irradiating the object with pulsed lights)

In this step, pulsed lights are irradiated from the light irradiation unit onto the object.

(S200: Acoustic wave detecting step of detecting an acoustic wave by a detector that is moving relative to the object)

The object irradiated with pulsed lights at step S100 generates an acoustic wave. In this step, the acoustic wave detecting element detects this acoustic wave, converts it into an electrical signal, and stores it in a memory or the like. As mentioned above, it is preferable to use a plurality of elements to detect the acoustic wave at a plurality of positions.

The steps S100 and S200 are carried out at respective positions as the acoustic wave detector and the light irradiation unit are moved in synchronism. That is, the steps S100 and S200 are repeated until an acoustic wave is detected from the entire region that is the target of object information acquisition. Therefore, the movement of the acoustic wave detector and the light irradiation unit and the timing of light irradiation and acoustic wave detection must be controlled.

If the plurality of acoustic wave detecting elements are equally spaced apart by a certain pitch distance as shown in FIG. 2, the acoustic wave detector should preferably be moved by one pitch each in the direction of arrangement so that each element detects the acoustic wave at respective positions. It is also preferable to arrange the acoustic wave detecting elements two-dimensionally in an equally distanced grid and to detect photoacoustic waves by irradiation of a pulsed light every time the detector is moved by one pitch in the direction of arrangement.

(S300: Summed signal obtaining step of adding together detection signals obtained at the same position relative to the object)

In this step, detection signals that were obtained at the same position on the object are selected from those obtained at step S200 and stored in the memory and added together to generate a summed signal. Alternatively, instead of storing all the detection signals, calculation may be performed in real time every time signals are output.

More specifically, as the acoustic wave detector is moved by one pitch for each element and detects a photoacoustic wave at each position, a signal detected by an element A at Position r₍ᵢ₎ on the object, and a signal detected by an element B (next to the element A) that has moved to the same position r₍ᵢ₎ during the next light irradiation, are added together. Similarly, as shown by Equations (2) to (8), signals obtained at the same position are added together to generate a summed signal. The summed signal may be divided by the number of signals that were added together to generate an average signal at this stage.

(S400: Initial sound pressure obtaining step of obtaining an initial sound pressure of a region of interest of the object by using weighted summed signal)

In this step, the average signal is weighted, as shown by Equation (13), in accordance with the adding process at step S300 described above to calculate an initial sound pressure. “In accordance with the adding process” means that, the average signal is not just multiplied by a correction factor but may be multiplied, too, for example, by a weight in accordance with the number of signals added together for respective positions of the object.

(S500: Weighted light quantity obtaining step of obtaining a weighted quantity of light of the region of interest in view of the adding process in the summed signal obtaining step and the weighting process in the initial sound pressure obtaining step)

In this step, the quantity of light of the region of interest for each pulsed light emitted at step S100 is calculated, and a weighted quantity of light is calculated as shown by Equation (14), in view of the adding process at step S300 and the weighting process at step S400. For example, the light quantity may be multiplied by a gain, in accordance with the gain obtained by weighting when obtaining the initial sound pressure. This processing in accordance with the adding process is necessary because, in this embodiment, the number of signals added together when calculating the initial sound pressure increases with the number of elements multiplied by the number of times of light irradiation (acoustic wave detection).

(S600: Object information of the region of interest is acquired from the weighted initial sound pressure of the region of interest and the weighted light quantity)

In this step, the weighted initial sound pressure obtained at step S400 is divided by the weighted light quantity obtained at step S500 to compute an absorption coefficient. A distribution of object information is thereby obtained.

Object information herein referred to may be oxygen saturation levels calculated based on absorption coefficients determined with the use of light of various wavelengths corresponding to the respective absorption coefficients of oxyhemoglobin and reductive hemoglobin.

According to the present invention, absorption coefficients can be calculated with better accuracy in photoacoustic imaging.

While the present invention has been described with reference to exemplary embodiments, it is to be understood that the invention is not limited to the disclosed exemplary embodiments. The scope of the following claims is to be accorded the broadest interpretation so as to encompass all such modifications and equivalent structures and functions.

This application claims the benefit of Japanese Patent Application No. 2012-171050, filed on Aug. 1, 2012, which is hereby incorporated by reference herein in its entirety.

What is claimed is:

1. An object information acquiring apparatus, comprising:
an irradiation unit irradiating an object with light;
a detection unit including a plurality of elements each obtaining an acoustic wave generated from the object irradiated with light and converting the acoustic wave into a detection signal;
a detection unit moving mechanism moving the detection unit to the object relatively;
an adding unit selecting, from detection signals converted from acoustic waves obtained by the plurality of elements at respective positions as the detection unit is moved, detection signals converted from acoustic waves obtained from an identical position on the object, and adding together these detection signals to output a resultant summed signal;
an initial sound pressure computing unit computing an initial sound pressure of a region of interest inside the object from the summed signal;
a light quantity computing unit computing a quantity of light of the region of interest; and
an object information computing unit computing object information using the initial sound pressure and the light quantity, wherein
the light quantity computing unit gives weight to a light quantity in accordance with processing in the adding unit and calculation processing in the initial sound pressure computing unit, to compute a weighted quantity of light.

2. The object information acquiring apparatus according to claim 1, wherein the light quantity computing unit computes the light quantity every time the detection unit obtains the acoustic wave.

3. The object information acquiring apparatus according to claim 1, wherein the initial sound pressure computing unit weights the summed signal in accordance with processing in the adding unit to compute a weighted initial sound pressure.

4. The object information acquiring apparatus according to claim 1, wherein the adding unit computes an average signal by dividing the summed signal of each position on the object by the number of signals added together for that position, and
the initial sound pressure computing unit weights each of the average signals for respective positions on the object in accordance with the number of signals added together for that position when computing the initial sound pressure.

5. The object information acquiring apparatus according to claim 1, wherein the adding unit weights and adds together detection signals converted from acoustic waves obtained from an identical position on the object to output a resultant summed signal.

6. The object information acquiring apparatus according to claim 1, further comprising an irradiation unit moving mechanism moving the irradiation unit on the object, wherein
the irradiation unit moving mechanism and the detection unit moving mechanism are operated in synchronism.

7. An object information acquiring method, comprising the steps of:
irradiating an object with light, by means of an irradiation unit;
obtaining an acoustic wave generated from the object irradiated with light and converting the acoustic wave into a detection signal, by means of a plurality of elements of a detection unit;
moving the detection unit on the object, by means of a detection unit moving mechanism;
selecting, from detection signals converted from acoustic waves obtained by the plurality of elements at respective positions as the detection unit is moved, detection signals converted from acoustic waves obtained from a relatively identical position to the object, and adding together these detection signals to output a resultant summed signal, by means of an adding unit;
computing an initial sound pressure of a region of interest inside the object from the summed signal, by means of an initial sound pressure computing unit;
computing a quantity of light of the region of interest, by means of a light quantity computing unit; and
computing object information using the initial sound pressure and the light quantity, by means of an object information computing unit, wherein
the light quantity computing unit gives weight to a light quantity in accordance with processing in the adding unit and calculation processing in the initial sound pressure computing unit, to compute the quantity of light.
